(19) United States
(12) Patent Application Publication
(10) Pub. No.: US 2025/0381129 A1
(43) Pub. Date: Dec. 18, 2025

(54) ANTI-AGING SKIN CARE COMPOSITION

(71) Applicant: L'OREAL, Paris (FR)

(72) Inventors: Alison Nicole TAYLOR, Jersey City, NJ (US); Julien LABOUREAU, Chevilly-Larue (FR); Saundra STRIHARSKY, Westtown, NY (US); I-Chien LIAO, Princeton, NJ (US); Patrick Morley WILLOUGHBY, New York, NY (US)

(73) Assignee: L'OREAL, Paris (FR)

(21) Appl. No.: 18/744,453

(22) Filed: Jun. 14, 2024

(51) Int. Cl. A61K 8/9789; A61K 8/365; A61K 8/67; A61Q 19/08

(52) U.S. Cl. CPC A61K 8/9789; A61K 8/365; A61K 8/671; A61Q 19/08

(57) ABSTRACT

A skin care composition includes a plurality of agents that include at least one retinol derivative and at least one bark extract, the plurality of agents providing reduced retinol associated inflammation as compared with a composition that includes the at least one retinol and lacks the at least one bark extract. The plurality of agents may include at least one retinol derivative comprising hydroxypinacolone retinoate and at least one bark extract which may include at least one bark extract comprising Eperua falcata bark extract. The plurality of agents may also include at least one hydroxy acid, for example, comprising lactic acid. The composition confers one or more of increased skin cell proliferation, improved cellular migration, improved cellular differentiation, or a combination thereof. Application the plurality of agents confers to skin cells and tissues an anti-inflammatory retinol genetic signature as demonstrated by via bulk-RNA sequencing of human keratinocytes and restores epidermal disruption and reduce IL-1A and IL-23 inflammatory cytokines in human ex-vivo skin models of chronic inflammation.

### FIELD OF THE INVENTION

The present invention is directed to a stable skin care composition having a combination of active components in amounts sufficient to address the signs of aging including a combination of ingredients that provide retinol-like efficacy without irritation and inflammation associated with retinols.

### BACKGROUND OF THE INVENTION

Anti-aging skin care compositions typically include a variety of active ingredients that confer various benefits to skin. In particular, retinols are beneficial conferring regeneration and thickening of the human epidermis by stimulating proliferation of keratinocytes which leads to the replacement and turnover of existing cells. Given the rapid turnover and shedding of the epithelium, a common side-effect of retinols is inflammation, which can be intolerable in populations of individuals with sensitized skin conditions, including chronic inflammation, atopic dermatitis, and psoriasis.

Thus, novel cosmetic formulations which stimulate epidermal renewal while reducing skin irritation are required for populations experiencing inflammatory and sensitive skin conditions.

### BRIEF SUMMARY OF THE INVENTION

In various embodiments, the disclosure provides a composition that includes a retinol derivative in combination with ingredients that reduce or eliminate the undesirable inflammatory issues associated with retinols. In various embodiments the composition includes at least one retinol derivative, at least one bark extract, and in some embodiments at least one hydroxy acid. In some embodiments, the bark extract includes one or more of at least one tannin and at least one flavonoid.

In the various embodiments, the composition may be provided in one of a variety of formulation types, for example, selected from an oil in water emulsion, a silicone in oil emulsion, a water in oil emulsion, or an anhydrous/glycolic formulation which may be lacking or substantially free of water (other than as a solvent for a raw material). In some anhydrous and/or glycol containing embodiments that have little water or are essentially free of water, glycols are present in amounts suitable to solubilize certain actives, such as niacinamide, in an otherwise anhydrous format.

The composition is stable and has a pleasing tactile feel.

In an embodiment, provided is a skin care composition comprising a plurality of agents that include at least one retinol derivative and at least one bark extract or plant extract with a high concentration of dihydroflavonols and catechuic tannins, the plurality of agents providing reduced retinol associated inflammation as compared with a composition that includes the at least one retinol and lacks the at least one bark extract, wherein the plurality of agents is selected from the group consisting of:
i. at least one retinol derivative comprising hydroxypinacolone retinoate and at least one bark extract or plant extract with a high concentration of dihydroflavonols and catechuic tannins;
ii. at least one retinol derivative comprising hydroxypinacolone retinoate, at least one bark extract or plant extract with a high concentration of dihydroflavonols and catechuic tannins, and at least one hydroxy acid; and
iii. at least one retinol derivative comprising hydroxypinacolone retinoate, at least one bark extract comprising Eperua falcata bark extract, and at least one hydroxy acid comprising lactic acid;
wherein the composition confers one or more of increased skin cell proliferation, improved cellular migration, improved cellular differentiation, or a combination thereof, and wherein application the plurality of agents confers to skin cells and tissues an anti-inflammatory retinol genetic signature as demonstrated by via bulk-RNA sequencing of human keratinocytes and restores epidermal disruption and reduce IL-1A and IL-23 inflammatory cytokines in human ex-vivo skin models of chronic inflammation.

In an embodiment, provided is a method for minimizing the signs of aging in skin, the method comprising:
(A) providing a plurality of agents that include at least one retinol derivative and at least one bark extract, the plurality of agents providing reduced retinol associated inflammation as compared with a composition that includes the at least one retinol and lacks the at least one bark extract, wherein the plurality of agents is selected from the group consisting of:
   i. at least one retinol derivative comprising hydroxypinacolone retinoate and at least one bark extract;
   ii. at least one retinol derivative comprising hydroxypinacolone retinoate, at least one bark extract, and at least one hydroxy acid; and
   iii. at least one retinol derivative comprising hydroxypinacolone retinoate, at least one bark extract comprising Eperua falcata bark extract, and at least one hydroxy acid comprising lactic acid;
wherein the composition confers one or more of increased skin cell proliferation, improved cellular migration, improved cellular differentiation, or a combination thereof;
(B) applying the composition to skin; and
(C) following a regimen of repeated application of the composition for a period of at least 1 day,
   wherein application the plurality of agents confers to skin cells and tissues an anti-inflammatory retinol genetic signature as demonstrated by via bulk-RNA sequencing of human keratinocytes and restores epidermal disruption and reduce IL-1A and IL-23 inflammatory cytokines in human ex-vivo skin models of chronic inflammation.

In some embodiments, the method includes providing one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

In an embodiment, provided is a skin care composition, comprising:
an anti-aging system, comprising:
   i. at least one retinol derivative;
   ii. at least one hydroxy acid; and
   iii. at least one bark extract.

In some embodiments of the skin care composition, in the anti-aging system:
i. the at least one retinol derivative is selected from the group consisting of hydroxypinacolone retinoate, retinol, retinyl acetate, retinyl palmitate, retinyl propionate, retinyl retinoate, bakuchiol, and combinations thereof;
ii. the at least one hydroxy acid is selected from the group consisting of lactic acid, gluconolactone, glycolic acid, mandelic acid, salicylic acid, and combinations thereof; and
iii. the at least one bark extract is selected from the group consisting of Eperua falcata bark extract, pine bark extract (Pinus pinaster bark/bud extract), Ficus regligiosa bark, Witch Hazel Extract, a plant extract with a high concentration of dihydroflavonols and catechuic tannins, and combinations thereof.

In some embodiments of the skin care composition, in the anti-aging system:
i. the at least one retinol derivative comprises hydroxypinacolone retinoate;
ii. the at least one hydroxy acid comprises lactic acid or gluconolactone; and
iii. the at least one bark extract comprises Eperua falcata bark extract.

In some embodiments of the skin care composition, in the anti-aging system;
i. the at least one retinol derivative is present from about 0.001% to about 3.0%,
ii. the at least one hydroxy acid is present from about 0.001% to about 5.0%; and
iii. the at least one bark extract is present from about 0.0001% to about 70%,
all amounts by weight, based on the total weight of the composition.

In some embodiments of the skin care composition, in the anti-aging system;
i. the at least one retinol derivative comprises hydroxypinacolone retinoate present from about 0.05% to about 0.5%;
ii. the at least one hydroxy acid comprises lactic acid present from about 1.0% to about 5.0%; and
iii. the at least one bark extract comprises Eperua falcata bark extract present from about 0.05% to about 0.5%,
all amounts by weight, based on the total weight of the composition.

In some embodiments of the skin care composition, in the anti-aging system:
i. hydroxypinacolone retinoate is present at about 0.1%;
ii. lactic acid is present at about 3%; and
iii. Eperua falcata bark extract is present at about 0.1%,
all amounts by weight, based on the total weight of the composition.

In some embodiments of the skin care composition, the composition comprises:
a carrier system comprising:
   i. at least one surfactant;
   ii. at least one fatty compound; and
   iii. at least one polymeric thickener.

In some embodiments of the skin care composition, the at least one surfactant is selected from the group consisting of glyceryl stearate (and) PEG-100 stearate, steareth-2, steareth-20, methyl gluceth-20, glyceryl stearate, and combinations thereof, the at least one fatty compound is selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof, and the at least one polymeric thickener is selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof.

In some embodiments of the skin care composition, the carrier system comprising:
i. water, a water-based solvent, or a combination thereof; and
ii. optionally, one or more additives selected from the group consisting of niacinamide, tocopherol, tocopheryl acetate, tocopherol, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, tetrasodium glutamate diacetate, sodium hydroxide, sodium PCA, phenoxyethanol, and combinations thereof.

In an embodiment, provided is a skin care composition, comprising:
an anti-aging system, comprising:
   i. at least one retinol derivative;
   ii. at least one hydroxy acid; and
   iii. at least one bark extract; and
a carrier system comprising:
   i. at least one surfactant;
   ii. at least one fatty compound;
   iii. at least one polymeric thickener;
   iv. water, a water-based solvent, or a combination thereof; and
   v. optionally, one or more additives.

In some embodiments of the skin care composition:
in the anti-aging system:
   i. the at least one retinol derivative is selected from the group consisting of hydroxypinacolone retinoate, retinol, retinyl acetate, retinyl palmitate, retinyl propionate, retinyl retinoate, bakuchiol, and combinations thereof;
   iv. the at least one hydroxy acid is selected from the group consisting of lactic acid, gluconolactone, glycolic acid, mandelic acid, salicylic acid, and combinations thereof; and
   ii. the at least one bark extract is selected from the group consisting of Eperua falcata bark extract, pine bark extract (Pinus pinaster bark/bud extract), Ficus regligiosa bark, Witch Hazel Extract, a plant extract with a high concentration of dihydroflavonols and catechuic tannins, and combinations thereof; and
in the carrier system:
   the at least one surfactant is selected from the group consisting of glyceryl stearate (and) PEG-100 stearate, steareth-2, steareth-20, methyl gluceth-20, glyceryl stearate, and combinations thereof;
   the at least one fatty compound is selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof; and
   the at least one polymeric thickener is selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof.

In some embodiments of the skin care composition, the composition comprises at least one additive selected from the group consisting of niacinamide, tocopherol, tocopheryl acetate, tocopherol, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, tetrasodium glutamate diacetate, sodium hydroxide, sodium PCA, phenoxyethanol, and combinations thereof.

The composition according to claim 13, wherein in the carrier system:
i. the composition comprises water present in a range from about from about 25% to about 75%;
ii. the at least one surfactant is present in a range from about 0.5% to about 3%;
iii. the at least one fatty compound is present in a range from about 0.1% to about 20%; and
iv. the at least one polymer thickener is present in a range from about 0.01% to about 10%,
all amounts by weight, based on the total weight of the composition.

In an embodiment, provided is a skin care composition, comprising:
an anti-aging system, comprising:
   i. at least one retinol derivative comprising hydroxypinacolone retinoate present from about 0.05% to about 0.5%; and
   ii. at least one bark extract comprising Eperua falcata bark extract present from about 0.05% to about 0.5%,
all amounts by weight, based on the total weight of the composition.

In some embodiments of the skin care composition, the composition comprises:
a carrier system comprising:
   i. at least one surfactant;
   ii. at least one fatty compound; and
   iii. at least one polymeric thickener.

In some embodiments of the skin care composition, the at least one surfactant is selected from the group consisting of glyceryl stearate (and) PEG-100 stearate, steareth-2, steareth-20, methyl gluceth-20, glyceryl stearate, and combinations thereof, the at least one fatty compound is selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof, and the at least one polymeric thickener is selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof.

The composition according to claim 18, the carrier system comprising:
i. water, a water-based solvent, or a combination thereof; and
ii. optionally, one or more additives selected from the group consisting of niacinamide, tocopherol, tocopheryl acetate, tocopherol, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, tetrasodium glutamate diacetate, sodium hydroxide, sodium PCA, phenoxyethanol, and combinations thereof.

Other features and advantages of the present invention will be apparent from the following more detailed description of the preferred embodiment which illustrates, by way of example, the principles of the invention.

### DESCRIPTION OF THE DRAWINGS

This application contains at least one drawing executed in color. Copies of this patent or patent application publication with color drawing(s) will be provided by the Office upon request and payment of the necessary fee.

FIG. 1A & B, wherein A is a schematic depicting epidermal turnover and wherein B is a schematic of In Vitro Pipeline;

FIG. 1C & C′, wherein C shows results in MTT (quantitative and sensitive detection of cell proliferation as it measures the growth rate of cells) Proliferation Assay and wherein C′ shows results in MTT Proliferation Assay;

FIG. 1D. shows results in Unbiased Gene Ontology Pathway Analysis;

FIG. 1E & E′, wherein each of E and E′ shows gene expression results;

FIG. 1E″ shows gene expression results;

FIG. 2A-A′ & B, wherein A is a schematic of the hallmarks of skin inflammation and wherein A′ is a schematic of nuclear morphology of infiltrating immune cells yellow boxes and wherein B is a schematic of experimental design;

FIG. 2C shows images of histological sections of human ex-vivo skin;

FIG. 2D & D′, wherein D shows quantification of secreted IL-1A and wherein D′ shows quantification of secreted IL-23;

FIG. 3A shows images of histological sections of human ex-vivo skin;

FIG. 3B & B′, wherein B shows quantification of secreted IL-1A and wherein B′ shows quantification of secreted IL-23.

It is to be understood that the foregoing and following descriptions are exemplary and explanatory only, and are not intended to be restrictive of any subject matter claimed.

### DETAILED DESCRIPTION OF THE INVENTION

According to the disclosure, the inventors sought to provide a skin care composition that provides the skin renewal benefits of retinols without the adverse effects associated with retinols including inflammation and related cellular and tissue effects.

Retinols regenerate and thicken the human epidermis by stimulating proliferation of keratinocytes which leads to the replacement and turnover of existing cells. Given the rapid turnover and shedding of the epithelium, a common side-effect of retinols is inflammation, which can be intolerable in populations with sensitized skin conditions including chronic inflammation, atopic dermatitis, and psoriasis, and those with fair skin. Thus, novel cosmetic formulations which stimulate epidermal renewal while reducing skin irritation are required, particularly for populations experiencing inflammatory and sensitive skin conditions.

The inventors demonstrated the surprising synergistic effects with novel combinations of the retinol, hydroxypinacolone retinoate (HPR), and at least one bark extract, for example, Eperua falcata bark extract (Eperuline), and in some embodiments, at least one hydroxy acid, for example, lactic acid (LA). The demonstrated results were obtained using human 2D keratinocytes and ex-vivo human skin. The inventors demonstrated that 2D Keratinocyte cell proliferation was stimulated through the pairing of HPR with LA while this effect was not seen with the poly hydroxy acid gluconolactone. The inventors demonstrated with RNA sequencing in 2D human keratinocytes that HPR-LA-Eperuline combinations exhibit an anti-inflammatory retinol genetic signature via bulk-RNA sequencing and that the specific combinations of HPR-Eperuline and HPR-LA-Eperuline restore epidermal disruption and reduce IL-1A and IL-23 inflammatory cytokines in human ex-vivo skin models of chronic inflammation. More specifically, referring to the data provided herein, when HPR-LA was paired with the anti-inflammatory extract Eperuline, treated keratinocytes contained a retinol-based genetic signature with reduced inflammatory profiles, wherein expression of IL-1A, IL-1B and IL-23 were demonstrated to be downregulated. Notably, IL-1A and IL-23 are associated with clinical manifestations of skin irritation and inflammation. Consistent with these data, when chronic inflammation was modelled in human ex-vivo skin, the inventors similarly found that Eperuline paired with HPR or HPR-LA reduced histological signs of inflammation and reduced secreted IL-1A and IL-23.

The unexpected findings with the novel combinations of HPR-Eperuline and HPR-LA-Eperuline provides a basis for retinol based anti-aging interventions with reduced inflammatory side-effects. Thus, these proposed combinations are likely best suited for consumer populations with sensitive skin.

### Composition

A skin care composition according to the disclosure includes a plurality of agents that include at least one retinol derivative and at least one bark extract, the plurality of agents providing reduced retinol associated inflammation as compared with a composition that includes the at least one retinol and lacks the at least one bark extract. In some embodiments, the plurality of agents includes at least one hydroxy acid, for example, lactic acid. Accordingly, the plurality of agents may include at least one retinol derivative comprising hydroxypinacolone retinoate and at least one bark extract which may include at least one bark extract comprising Eperua falcata bark extract, and in some embodiments, at least one hydroxy acid, for example, comprising lactic acid. The composition confers one or more of increased skin cell proliferation, improved cellular migration, improved cellular differentiation, or a combination thereof. Application the plurality of agents confers to skin cells and tissues an anti-inflammatory retinol genetic signature as demonstrated by via bulk-RNA sequencing of human keratinocytes and restores epidermal disruption and reduce IL-1A and IL-23 inflammatory cytokines in human ex-vivo skin models of chronic inflammation.

The skin care composition according to the present disclosure finds its application in a wide variety of treatments, especially cosmetic treatments of the keratinous tissue, such as skin, and most particularly facial skin. Of course, it will be appreciated that while the inventive composition is particularly suited for use on facial skin, it may be beneficial for use on specific areas of facial skin and on the body more generally, for example on skin on the lips, neck, hands, abdominal, and the hair, including the scalp.

As described herein, in some embodiments, the skin care composition may be used according to a monthly treatment regimen or may be used for daily application over a shorter or longer period of time. In addition, the skin care composition may be used in conjunction with one or more applicators and instruments, and optionally as part of a more expansive treatment regimen that includes use of compositions with other actives and/or use of one or more applicators and instruments.

In some embodiments, the skin care composition may be used with one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

The various components of the composition as provided herein below represent exemplary embodiments and various alternate embodiments are given as examples, thus, it will be appreciated that other optional components compatible with cosmetic applications known in the art may be used. And of course, any one of the possible components may be excluded.

### Retinol Derivative

In various embodiments, the composition includes at least one retinol derivative selected from the group consisting of hydroxypinacolone retinoate, retinol, retinyl acetate, retinyl palmitate, retinyl propionate, retinyl retinoate, bakuchiol, and combinations thereof.

Generally, suitable retinol derivatives can be selected from the group consisting of hydroxypinacolone retinoate, retinol, retinyl acetate, retinyl palmitate, retinyl propionate, retinyl retinoate, bakuchiol, and combinations thereof. Retinol derivatives may also be referred to as retinal, retinal esters, or retinyl linoleate.

In some embodiments, the at least one retinol derivative includes hydroxypinacolone retinoate.

In some embodiments, the skin care composition is in the form of an oil and water emulsion.

### Hydroxypinacolone Retinoate

In various embodiments, the skin care composition includes at least one retinol derivative. In some embodiments, the skin care composition includes at least the retinol derivative hydroxypinacolone retinoate.

The amount of the at least one retinol derivative, for example, hydroxypinacolone retinoate, present in the skin care composition will vary but in various embodiments it is from about 0.001% to about 3%, or from about 0.01% to about 0.5%, or from about 0.05% to about 0.2%, or about 0.1%, or any suitable combination, sub-combination, range, or sub-range thereof by weight, based on the weight of the skin care composition. One of ordinary skill in the art, however, will appreciate that other ranges are within the scope of the invention.

Thus, the at least one retinol derivative, for example, hydroxypinacolone retinoate, is present, by weight, based on the total weight of the skin care composition, from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, to about 3 weight percent, including increments and ranges therein and there between.

### Bark Extract

In various embodiments, the composition includes at least one bark extract selected from the group consisting of Eperua falcata bark extract, pine bark extract (Pinus pinaster bark/bud extract), Ficus regligiosa bark, and combinations thereof. In some embodiments, the composition includes as the bark extract any natural plant extract that is high in dihydroflavonols and catechuic tannins.

In some embodiments, the skin care composition is in the form of an oil and water emulsion.

Eperua falcata Bark Extract

In some embodiments, the at least one anti-inflammatory includes Eperua falcata bark extract. Eperua falcata bark extract reduces ROS, reduces inflammation and enhances mitophagy in skeletal muscle.

When present, the amount of the at least one anti-inflammatory agent, for example, Eperua falcata bark extract, in the skin care composition will vary but in various embodiments it is from about 0.01% to about 10%, or from about 0.02% to about 5%, or from about 0.04% to about 0.5%, or from about 0.06% to about 0.2%, or about 0.1%, or any suitable combination, sub-combination, range, or sub-range thereof by weight, based on the weight of the skin care composition. One of ordinary skill in the art, however, will appreciate that other ranges are within the scope of the invention.

Thus, at least one anti-inflammatory agent, for example comprising Eperua falcata bark extract, is present, by weight, based on the total weight of the skin care composition, from about 0.01, 0.02, 0.03, 0.04, 0.05, 0.06, 0.07, 0.08, 0.90, 0.1, 0.2, 0.3, 0.4, 0.5, 0.6, 0.7, 0.8, 0.9, 1.0, 2, 3, 4, 5, 6, 7, 8, 9, to about 10 weight percent, including increments and ranges therein and there between.

### Hydroxy Acid

In various embodiments, the composition may include at least one hydroxy acid.

In some embodiments, the composition includes at least one alpha hydroxy acid comprising lactic acid or one poly hydroxy acid comprising gluconolactone. In some embodiments, the composition includes at least lactic acid.

### Alpha Hydroxy Acids

In accordance with some embodiments, the skin care composition may comprise at least one alpha hydroxy acid. In some embodiments, the composition includes at least one alpha hydroxy acid comprising lactic acid. Lactic acid, or 2-hydroxypropanoic acid, may provide enhanced exfoliation of the skin and may boost production of glycosaminoglycan (GAG) in the skin, improving the barrier function and moisturization of skin.

Suitable alpha hydroxy acids include lactic acid, glycolic acid, tartaric acid, mandelic acid, citric acid, ester derivatives of the foregoing, and combinations thereof. Exemplary ester derivatives include ester compounds of glycolic acid, lactic acid, such as methyl lactate, ethyl lactate, butyl lactate and, similarly, ester compounds of glycolic acid, tartaric acid, mandelic acid, citric acid.

When present, the skin care composition includes a concentration of alpha hydroxy acid in a range from about 0.0001% to about 70%, or from about 0.001% to about 60%, or from about 0.01% to about 50%, or from about 0.1% to about 25%, or from about 1% to about 15%, or from about 1% to about 5%, or about 3%, or any suitable combination, sub-combination, range, or sub-range thereof by weight, based on the weight of the skin care composition. One of ordinary skill in the art, however, will appreciate that other ranges are within the scope of the invention. In accordance with some embodiments, the amount of alpha hydroxy acid present is not more than about 10%.

Thus, any one of or a combination of alpha hydroxy acid may be present, by weight, based on the total weight of the skin care composition, from about 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 56, 57, 58, 59, 60, 61, 62, 63, 64, 65, 66, 67, 68, 69 to about 70 weight percent, including increments and ranges therein and there between.

### Poly Hydroxy Acid

In accordance with some embodiments, the skin care composition may comprise at least one poly hydroxy acid.

In some embodiments, the composition includes at least one poly hydroxy acid comprising gluconolactone. Suitable poly hydroxy acids include gluconolactone and derivatives thereof and combinations thereof.

When present, the skin care composition includes a concentration of poly hydroxy acid in a range from about 0.0001% to about 70%, or from about 0.001% to about 60%, or from about 0.01% to about 50%, or from about 0.1% to about 25%, or from about 1% to about 15%, or from about 1% to about 5%, or about 3%, or any suitable combination, sub-combination, range, or sub-range thereof by weight, based on the weight of the skin care composition. One of ordinary skill in the art, however, will appreciate that other ranges are within the scope of the invention. In accordance with some embodiments, the amount of poly hydroxy acid present is not more than about 10%.

Thus, any one of or a combination of poly hydroxy acid, when present, is present, by weight, based on the total weight of the skin care composition, from about 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 56, 57, 58, 59, 60, 61, 62, 63, 64, 65, 66, 67, 68, 69 to about 70 weight percent, including increments and ranges therein and there between.

### Beta Hydroxy Acid

In accordance with some embodiments, the skin care composition may comprise at least one beta hydroxy acid. The term “beta-hydroxy acid” is understood to mean a carboxylic acid having a hydroxyl functional group and a carboxylic functional group separated by two carbon atoms. A beta hydroxy acid can be present in the skin care composition in the form of the free acid and/or in the form of one of its associated salts (salts with an organic base or an alkali metal, in particular), especially according to the final pH imposed on the skin care composition.

Suitable beta hydroxy acids include salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid, and combinations of these.

When present, the skin care composition includes a concentration of beta hydroxy acid in a range from about 0.1% up to and not more than about 2% of beta hydroxy acid, or about 1.9% of beta hydroxy acid, or up to and not more than about 1% of beta hydroxy acid, or from about 0.1% to about 1% of beta hydroxy acid, or from about 0.2% to about 2.0%, or from about 0.1% to about 1.5%, or from about 0.2% to about 1.5%, or from about 0.3% to about 1.0%, or from about 0.35% to about 0.75%, or from about 0.4% to about 0.5%, or is about 0.45%, or any suitable combination, sub-combination, range, or sub-range thereof by weight, based on the weight of the skin care composition. One of ordinary skill in the art, however, will appreciate that other ranges are within the scope of the invention.

Thus, the at least one beta hydroxy acid, when present, is present in the skin care composition, from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

### Surfactant

In various embodiments, the skin care composition may comprise at least one surfactant selected from nonionic, cationic, amphoteric, and anionic surfactants. In some embodiments, the skin care composition may include, or alternatively may be substantially free from, or exclude any one or more of nonionic, cationic, amphoteric, or anionic surfactants.

In some embodiments, the composition may include at least one surfactant selected from the group consisting of glyceryl stearate (and) PEG-100 stearate, steareth-2, steareth-20, methyl gluceth-20, glyceryl stearate, and combinations thereof.

In some embodiments, the composition may include each one of glyceryl stearate (and) PEG-100 stearate, steareth-2, steareth-20, methyl gluceth-20, glyceryl stearate, and combinations thereof.

In some embodiments, one or more surfactants may be provided incidentally as carriers in raw materials containing selected actives or additives, and such surfactants are not added specifically as a component of the inventive composition.

When present, the at least one surfactant is present in the skin care composition from about 0.1% to about 15%, or from about 0.1% to about 12%, or from about 0.1% to about 10%, or from about 0.2% to about 5%, or from about 0.5% to about 3%, or any suitable combination, sub-combination, range, or sub-range thereof by weight, based on the weight of the skin care composition. One of ordinary skill in the art, however, will appreciate that other ranges are within the scope of the invention.

In some embodiments, the skin care composition includes more than one surfactant, wherein each surfactant may be present in the skin care composition from about 0.1% to about 15%, or from about 0.1% to about 12%, or from about 0.1% to about 10%, or from about 0.2% to about 5%, or from about 0.5% to about 3%, or any suitable combination, sub-combination, range, or sub-range thereof by weight, based on the weight of the skin care composition. According to such embodiments, the total amount of surfactant that may be present in the composition may be in a range from about 0.1% to about 25%, or from about 0.1% to about 15%, or from about 0.1% to about 10%, or from about 0.5% to about 9%, or any suitable combination, sub-combination, range, or sub-range thereof by weight, based on the weight of the skin care composition.

In some representative embodiments surfactants may be present in amounts as follows (all as percentages, (%) by weight, based on the weight of the composition): steareth-2 (0.50), steareth-20 (1.00), methyl gluceth-20 (1.00), glyceryl stearate (0), glyceryl stearate (1.50), PEG-100 stearate (1.50).

In some representative embodiments surfactants may be present in amounts as follows (all as percentages, (%) by weight, based on the weight of the composition): steareth-2 (1.50), steareth-20 (2.00), methyl gluceth-20 (0), glyceryl stearate (3.50), PEG-100 stearate (1.50).

Thus, the at least one surfactant, when present, is present, by weight, based on the total weight of the skin care composition, from about from about 0.1, 0.2, 0.3, 0.4, 0.5, 0.6, 0.7, 0.8, 0.9, 1, 2, 3, 4, 5, 6, 7, 8, 9, 10, 11, 12, 13, 14, to about 15 weight percent, including increments and ranges therein and there between, and a combination of surfactants, when present, are present, by weight, based on the total weight of the skin care composition, from about from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, to about 25 weight percent, including increments and ranges therein and there between.

### Fatty Compounds and Ceramides

In some embodiments, the composition may include at least one fatty compound.

In some embodiments, the composition may include fatty compounds selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof.

In some embodiments, the composition may include each of the fatty compounds comprising caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, and sodium lauroyl lactylate (and) ceramide NP (and) ceramide AP (and) phytosphingosine (and) cholesterol (and) xanthan gum (and) carbomer (and) ceramide EOP.

In some embodiments, the composition may include at least one fatty compound selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, one or more ceramides selected from selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, Phytosphingosine, Sphingosine, 2-Oleyl-1,3-octadecanediol, ceramide precursors, fatty acids, fatty alcohols, cholesterol, cholesterol sulfate and combinations thereof.

In some embodiments, one or more fatty compounds may be provided incidentally as carriers in raw materials containing selected actives or additives, and such fatty compounds are not added specifically as a component of the inventive composition.

When present, the at least one fatty compound is present in the skin care composition is from about 0.1% to about 20%, or from about 2% to about 18%, or from about 5% to about 15%, or from about 8% to about 12%, or from about 2% to about 6%, or from about 0.1% to about 3%, or from about 0.1% to about 0.35%, or from about 1% to about 3%, or from about 1.5% to about 2%, or about 0.1%, or about 0.35%, or about 1%, or about 1.5%, or about 2%, or about 3%, or any suitable combination, sub-combination, range, or sub-range thereof by weight, based on the weight of the skin care composition. One of ordinary skill in the art, however, will appreciate that other ranges are within the scope of the invention.

In some embodiments, the skin care composition includes more than one fatty compound, wherein each fatty compound may be present in the skin care composition in the foregoing stated ranges and amounts. According to such embodiments, the total amount of fatty compound that may be present in the composition may be in a range from about 0.1% to about 40%, or any suitable combination, sub-combination, range, or sub-range thereof by weight, based on the weight of the skin care composition.

In some embodiments, the total amount of fatty compounds includes from about 5% to about 30%, or from about 8% to about 24%, or from about 10% to about 20%, by weight, based on the total weight of the skin care composition.

In some representative embodiments fatty compounds may be present in amounts as follows (all as percentages, (%) by weight, based on the weight of the composition): bis-behenyl/isostearyl/phytosteryl dimer dilinoleyl dimer dilinoleate (3), Butyrospermum parkii (shea) butter (2), c15-19 alkane (1.00), ceramide ap (0.002750), ceramide eop (0.000005), ceramide np (0.005000), cholesterol (0.35), caprylic/capric triglyceride (2.00), dicaprylyl ether (0), dimethicone (0), dimethicone (3.36), Glycine soja (soybean) oil (0.05), glycol palmitate (1.50), squalane (3.00), polysilicone-11 (0.64).

In some representative embodiments fatty compounds may be present in amounts as follows (all as percentages, (%) by weight, based on the weight of the composition): bis-behenyl/isostearyl/phytosteryl dimer dilinoleyl dimer dilinoleate (0), Butyrospermum parkii (shea) butter (0), C15-19 alkane (3.00), ceramide ap (0.002750), ceramide eop (0.000005), ceramide np (0.005000), cholesterol (0.10), caprylic/capric triglyceride (0), dicaprylyl ether (2), dimethicone (0.75), dimethicone (0), Glycine soja (soybean) oil (0), glycol palmitate (0), squalane (2.00), linoleic acid (0.38), linolenic acid (0.12), polysilicone-11 (0).

Thus, the at least one fatty compound, when present, is present, by weight, based on the total weight of the skin care composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 weight percent, including increments and ranges therein and there between, and a combination of fatty compounds, when present, are present, by weight, based on the total weight of the skin care composition, from about from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40 weight percent, including increments and ranges therein and there between.

### Cosmetically Acceptable Solvent

Embodiments of the skin care composition according to the disclosure may contain at least one cosmetically acceptable solvent. In various embodiments, the cosmetically acceptable solvent may be chosen from water, water-based solvents, or combinations thereof. In some embodiments, the composition is anhydrous and includes one or more water-based solvents for solvating actives and additives. In some embodiments, the composition is an emulsion of the type water in oil, oil in water, or silicone in water, etc.

### Water

In accordance with the various embodiments, water may be present in the skin care composition in a range from about 10% to about 90%, or from about 20% to about 75%, or from about 25% to about 70%, or from about 30% to about 65%, or from about 50% to about 60%, or any suitable combination, sub-combination, range, or sub-range thereof by weight, based on the weight of the skin care composition. One of ordinary skill in the art, however, will appreciate that other ranges are within the scope of the invention.

Thus, water may be present by weight, based on the weight of the skin care composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60. 61, 62, 63, 64, 65, 66, 67, 68, 69, 70, 71, 72, 73, 74, 75, 76, 77, 78, 79, 80, 81, 82, 83, 84, 85, 86, 87, 88, 89 to about 90 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene.

The water phase may also comprise reconstituted thermal water, that is to say a water comprising trace elements such as zinc, copper, magnesium, etc., reconstituting the characteristics of a thermal water.

The skin care composition has a pH from about 3 to about 7, and in some embodiments from about 3.5 to about 7, and in some embodiments about 4 to about 5, or about 4.8.

The pH may be adjusted to the desired value by addition of a base (organic or inorganic), for example sodium hydroxide, potassium hydroxide, or another suitable base, or combinations thereof.

### Water-Soluble Solvents

In accordance with some embodiments, the skin care composition may include at least one water-based or water-soluble solvent. The terms “water-soluble solvent,” “water-miscible solvent” and “water-based solvent” and mean a compound that is liquid at 25° C. and at atmospheric pressure (760 mmHg), and it has a solubility of at least 50% in water under these conditions. In some cases, the water-based solvent has a solubility of at least 60%, 70%, 80%, or 90% in water under these conditions.

In some embodiments, the composition includes water and at least one water-based solvent. In some embodiments, the composition does not include water or any water-based solvents. In some embodiments, the composition is free of water and includes water-based solvents, for example glycols.

In some embodiments, the composition includes at least one water based solvent selected from the group consisting of water, glycerin, propanediol, butylene glycol, pentylene glycol, dipropylene glycol, propylene glycol, and combinations thereof.

In some particular embodiments, the composition includes water, glycerin, propanediol, and butylene glycol.

In some embodiments, water or one or more water-based solvents may be provided incidentally as carriers in raw materials containing selected actives or additives, and such solvents are not added specifically as a component of the inventive composition.

As examples of organic solvents, non-limiting mentions can be made of ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. The organic solvents can be volatile or non-volatile compounds.

Further non-limiting examples of water-based solvents include alkanols (polyhydric alcohols, glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-hexanediol, 1,2-pentanediol, and 4-methyl-1,2-pentanediol.

Further non-limiting examples of water-based solvents include alkyl alcohols having 1to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments, when present, the amount of the at least one water-based solvent is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the skin care composition. One of ordinary skill in the art, however, will appreciate that other ranges are within the scope of the invention.

In some embodiments, the skin care composition includes more than one water soluble solvent, each water soluble solvent present in an amount as set forth herein above, wherein each different water soluble solvent may be present within one of the ranges selected from the ranges set forth herein above.

In some representative embodiments water based solvents may be present in amounts as follows (all as percentages, (%) by weight, based on the weight of the composition): glycerin (4.6), propanediol (0), butylene glycol (3.60), ethylhexylglycerin (0.0015).

In some representative embodiments water based solvents may be present in amounts as follows (all as percentages, (%) by weight, based on the weight of the composition): glycerin (6.7), propanediol (2.00), butylene glycol (0.60), ethylhexylglycerin (0.0015).

Thus, each one or combination of water-based solvents may be present by weight, based on the total weight of the skin care composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Polymers/Thickeners

In various embodiments, the skin care composition may include at least one thickener. The thickener may be thickening polymers.

In some embodiments, the at least one thickener is a polymer selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof.

In some embodiments, the at least one thickening polymer includes each of xanthan gum, poly C10-30 alkyl acrylate, polyacrylate crosspolymer-6, acrylates/C10-30 alkyl acrylate crosspolymer.

In some embodiments, one or more thickening polymers may be provided incidentally as carriers in raw materials containing selected actives or additives, and such thickening polymers are not added specifically as a component of the inventive composition.

Other non-limiting examples of thickening polymers may be selected from the group consisting of sclerotium gum, xanthan gum, carrageenan, acacia, agar, algin, alginic acid, ammonium alginate, amylopectin, calcium alginate, sodium alginate, calcium carrageenan, carnitine, carrageenan, dextrin, gelatin, gellan gum, guar gum, tragacanth gum, acacia gum, Arabic gum, guar hydroxypropyltrimonium chloride, hectorite, hydrated silica, hydroxypropyl chitosan, hydroxypropyl guar, karaya gum, kelp, locust bean gum, natto gum, potassium alginate, potassium carrageenan, propylene glycol alginate, sodium carboxymethyl dextran, sodium carrageenan, tragacanth gum, modified xanthan gum, biosacharide gum, chitin, levan, elsinan, collagen, zein, gluten, soy protein, casein, Zea mays (corn) starch, poly C10-30 alkyl acrylate, ammonium acryloyldimethyltaurate/VP copolymer, carbomer, and combinations thereof.

The at least one thickening polymer, when present, is present in the skin care composition from about 0.01% to about 10%, or from about 0.2% to about 3%, or from about 0.5% to about 2%, or from about 0.2% to about 0.8%, or from about 0.3% to about 0.5%, or any suitable combination, sub-combination, range, or sub-range thereof by weight, based on the weight of the skin care composition. One of ordinary skill in the art, however, will appreciate that other ranges are within the scope of the invention.

In some embodiments, the skin care composition includes more than one thickening polymer, wherein each thickening polymer may be present in the skin care composition in the foregoing stated ranges and amounts. According to such embodiments, the total amount of thickening polymer that may be present in the composition may be in a range from about 0.1% to about 20%, from about 0.2% to about 10%, or from about 0.5% to about 3%, or from about 0.8% to about 2%, or about 1%, or up to about 1%, or up to about 2%, or any suitable combination, sub-combination, range, or sub-range thereof by weight, based on the weight of the skin care composition.

In some representative embodiments polymers may be present in amounts as follows (all as percentages, (%) by weight, based on the weight of the composition): acrylates/C10-30 alkyl acrylate crosspolymer (0.4), xanthan gum (0.30175), poly C10-30 alkyl acrylate (0), polyacrylate crosspolymer-6 (0.30), carbomer (0.0315).

In some representative embodiments polymers may be present in amounts as follows (all as percentages, (%) by weight, based on the weight of the composition): acrylates/C10-30 alkyl acrylate crosspolymer (0.50), xanthan gum (0.00175), poly C10-30 alkyl acrylate (0.60), polyacrylate crosspolymer-6 (0.30), carbomer (0.0315).

Thus, one or a combination of thickener may be present, by weight, based on the total weight of the skin care composition, from about 0.01, 0.02, 0.03, 0.04, 0.05, 0.06, 0.07, 0.08, 0.90, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, to about 20 weight percent, including increments and ranges therein and there between.

### Optional Additives

In some embodiments, there may be one or more optional actives or other ingredients (herein, “additives”) present in the skin care composition.

In some embodiments, one or more additives may be provided incidentally as carriers in raw materials containing selected actives or additives, and such additives are not added specifically as a component of the inventive composition. Accordingly, in some embodiments, the skin care composition includes incidental additives (which may include, generally, additives as described herein below, as well as surfactants, fatty compounds, solvents, and polymeric thickeners) that are present in the raw materials of various ingredients as including in the inventive composition. For example, ceramides as may be included in the inventive skin care composition include the ceremide actives together with other ingredients that include sodium lauroyl lactylate, phytosphingosine, cholesterol, xanthan bum, and carbomer. Other incidental ingredients include, but are not limited to, dextrin, dimethyl isosorbide, and sodium lactate.

In some embodiments, the skin care composition includes at least one additive that is a skin active selected from the group consisting of niacinamide, tocopherol, tocopheryl acetate, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, and combinations thereof.

In some embodiments, the skin care composition includes additives selected from the group consisting of tetrasodium glutamate diacetate, sodium hydroxide, sodium PCA, phenoxyethanol, and combinations thereof.

In some embodiments, the skin care composition includes each of niacinamide, tocopherol, tocopheryl acetate, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, and tetrasodium glutamate diacetate, sodium hydroxide, sodium PCA, and phenoxyethanol.

In some embodiments, the one or more additives may be selected from: anti-microbials; chelating agents; oils; fatty alcohols; fatty amides; alkylene carbonates; glycols; lower alcohols (e.g. ethanol; propanediol); anti-elastase and anti-collagenase agents; peptides; fatty acid derivatives; steroids; trace elements; extracts of algae and of planktons; enzymes and coenzymes; cationic polymers such as nature based polymers such as chitosan and polylysine, and polyquaternium compounds; fillers; clays; penetrants; sequestrants; fragrances; dispersants; skin care actives such as organic and inorganic UV filters.

In some embodiments, the one or more additives may be selected from citric acid; phenylethyl resorcinol; hydroxyacetophenone; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol or resveratrol glucoside, curcumin, pinoresinol, ferulic acid, hydroxytyrosol, cinnamic acid, caffeic acid, p-coumaric acid, baicalin (Scutellaria baicalensis root extract), ellagic acid; hyaluronic acid and its derivatives; escin (also known as Aescin, a mixture of saponins with anti-inflammatory, vasoconstrictor and vasoprotective effects found in Aesculus hippocastanum); niacinamide; jasmonic acid; {2-acetyl-3-trifluoromethylphenyl)amino)-3-methylbutyrylamino} acetic acid; phloretin; acetyl trifluoromethylphenyl valyl glycine; hesperidin or neohesperidin; biocellulose; vitamins and vitamin derivatives, such as vitamin C, ethylvitamin C, vitamin E (tocopherol); aloe vera; calmosensin; Inula helenium extract; Resargin; Andrographis paniculate extract; Ficus regligiosa bark; Cucuma zeoaria extract; Ursolic acid; ectoin; Coptidis chinensis; Pongamia pinnata extract; White Hibiscus; ectoin; Punica granatum; pomegranate extract; ginger root extract; ginkgo biloba extract; witch hazel; exosome; bisabolol (levomenol); green tea extract; zinc oxide; and combinations thereof.

Although the aforementioned optional additives are given as examples, it will be appreciated that other optional components compatible with cosmetic applications known in the art may be used. And of course, any one of the aforementioned additives may be excluded.

In accordance with the various embodiments, the amounts of additives, for example, actives and other components, that may be present in the skin care composition can range from about 0.001% to about 50%, or from about 0.5% to about 30%, or from about 1.5% to about 20%, and from about 5% to about 15%, or any suitable combination, sub-combination, range, or sub-range thereof by weight, based on the weight of the skin care composition.

In some representative embodiments additives and raw materials carriers/solvents may be present in amounts as follows (all as percentages, (%) by weight, based on the weight of the composition): camellia sinensis leaf extract (0.10), citric acid (0.00672), dextrin (0.90), dimethyl isosorbide (1.80), bisabolol (0.50), boron nitride (0.75), hydrolyzed sodium hyaluronate (0.20), linoleic acid (0.38), linolenic acid (0.12), niacinamide (1.00), palmitoyl tetrapeptide-7 (0.00015), palmitoyl tripeptide-1 (0.0003), panthenol (0.5025), pentaerythrityl tetra-di-t-butyl hydroxyhydrocinnamate (0.20), phenoxyethanol (0.70), phytosphingosine (0.00275), polysorbate 20 (0.015), sodium hydroxide (0.90), sodium lactate (0.03), sodium lauroyl lactylate (0.05), sodium pca (0.50), superoxide dismutase (0.001), tetrahexyldecyl ascorbate (2.00), tetrasodium glutamate diacetate (0.095), tocopherol (0), tocopherol (˜0.95), ubiquinone (0.03).

In some representative embodiments additives and raw materials carriers/solvents may be present in amounts as follows (all as percentages, (%) by weight, based on the weight of the composition): camellia sinensis leaf extract (0.10), citric acid (0.005), dextrin (0.90), dimethyl isosorbide (1.80), bisabolol (0.50), boron nitride (1.00), hydrolyzed sodium hyaluronate (0.20), niacinamide (1.50), palmitoyl tetrapeptide-7 (0.00015), palmitoyl tripeptide-1 (0.0003), panthenol (0.3750), pentaerythrityl tetra-di-t-butyl hydroxyhydrocinnamate (0.10), phenoxyethanol (0.70), phytosphingosine (0.00275), polysorbate 20 (0.015), sodium hydroxide (0.90), sodium lactate (0.03), sodium lauroyl lactylate (0.05), sodium pca (0.50), superoxide dismutase (0.001), tetrahexyldecyl ascorbate (0.25), tetrasodium glutamate diacetate (0.095), tocopherol (˜0.55), ubiquinone (0.03).

Thus, one or a combination of additives may be present in the skin care composition, by weight, based on the weight of the skin care composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

Of course, a person skilled in the art will take care to choose this or these optional additional compounds so that the advantageous properties intrinsically attached to the composition in accordance with the present disclosure are not, or not substantially, detrimentally affected by the envisaged addition or additions.

A person skilled in the art will take care to select this or of these optional additional compound(s), and/or the amount thereof, such that the advantageous properties of the composition according to the present disclosure are not, or are not substantially, adversely affected by the envisaged addition.

### Stability: Two Month Stable at 45° C.

Embodiments of the skin care composition according to the disclosure are considered a stable composition despite the high amounts of actives when they are shown to have maintained an aesthetically homogeneous phase, wherein there is no visually perceptible signs of phase separation, do not show a grainy texture and/or become inhomogeneous over a period of 8 weeks in a temperature storage range of 4° C. to 45° C., with no significant changes in appearance, pH, viscosity, and color.

### Pleasing Tactile Feel

Embodiments of the skin care composition according to the disclosure, include a pleasing tactile feel. By “pleasing tactile feel” and grammatical variations thereof, it is meant that embodiments of the skin care composition have a texture that is pleasing to the consumer and is substantially free of rough, clingy or sticky feel, and problems of innocuity with respect to the skin.

### Packaging

The skin care composition may be assembled into a kit or system including the skin care composition and at least one device or co-treatment device. The device or co-treatment device may include one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

### Definitions and Terms

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense. The term “comprising” is intended to be inclusive or open-ended and does not exclude any additional, unrecited element, method, step, or material.

The term “consisting of” excludes any element, step, or material other than those specified in the claim and, in the latter instance, impurities ordinary associated with the specified material(s).

The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point.

A range given of “about 3% to 7%” is intended to have the term “about” modifying both the 3% and the 7% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient in the raw material except as specifically stated. All amounts given herein are relative to the amount of active material, unless otherwise indicated.

All percentages, parts and ratios herein are based upon the total weight of embodiments of the skin care composition of the present disclosure, unless otherwise indicated.

As used herein, the term “surfactants,” as well as any specifically identified surfactants, includes salts of the surfactants even if not explicitly stated.

As used herein, the term “synthetic” means a material that is not of natural origin. The term “natural” and “naturally-sourced” and “nature-based” means a material of natural origin, such as derived from plants, which also cannot be subsequently chemically or physically modified. “Plant-based” means that the material came from a plant.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

As used herein, the term “substantially free” or “essentially free” as used herein means the specific material may be present in small amounts that do not materially affect the basic and novel characteristics of the Embodiments of the skin care composition according to the disclosure or the material may be absent. For instance, there may be less than 2% by weight of a specific material added to a composition, based on the total weight of the compositions (provided that an amount of less than 2% by weight does not materially affect the basic and novel characteristics of embodiments of the skin care composition according to the disclosure. Similarly, the compositions may include less than 2%, less than 1.5%, less than 1%, less than 0.5%, less than 0.1%, less than 0.05%, or less than 0.01%, or none (0%) of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

### EXAMPLES

The following examples are intended to be non-limiting and explanatory in nature only. In the Examples, amounts are expressed in percentage by weight (wt %) of active materials, relative to the total weight of the composition.

### EXAMPLE 1: Raw Materials

Percentages of each ingredient as may be exemplified in the following composition examples are shown as amount of actives, wherein the raw materials containing the active may be present in an amount that is equal to the amount of active, or if the raw material has a concentration of active that is less than 100%, then the cosmetic composition includes the raw material that includes active and a suitable solvent, wherein the concentration of active in the raw material is provided herein below in Table 1. When referring to the inventive and comparative examples, it should be presumed that each ingredient as shown in the exemplified compositions is final amount of active unless otherwise indicated, and not the amount of the RM that may contain less than 100% active.

TABLE 1
Select Raw Materials (including RMs having
active concentrations of less than 100%)

| RM | ~% Active In Rm |
|---|---|
| Lactic Acid | 90% |
| Palmitoyl Tripeptide -1 | <10% |
| Palmitoyl tetrapeptide-7 | <10% |
| Hydrodypinacolone retinoate | 10% |
| Eperua falcata bark extract | 10% |
| CERAMIDE EOP | <10% |
| CERAMIDE NP | <10% |
| CERAMIDE AP | <10% |

### EXAMPLE 2: Exemplary Embodiments of the Inventive Composition

Some exemplary embodiments of the inventive skin care composition according to the disclosure includes hydroxypinacolone retinoate, one of lactic acid or gluconolactone, and dextrin (and) Eperua falcata bark extract.

In some embodiments, the composition also includes, at least one surfactant selected from the group consisting of glyceryl stearate (and) PEG-100 stearate, steareth-2, steareth-20, methyl gluceth-20, glyceryl stearate, and combinations thereof, at least one fatty compound selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof, at least one polymer selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof, at least one additive that is a skin active selected from the group consisting of niacinamide, tocopherol, tocopheryl acetate, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, tetrasodium glutamate diacetate, sodium hydroxide, sodium PCA, phenoxyethanol, and combinations thereof, and water, glycerin, propanediol, and butylene glycol.

TABLE 2
Exemplary Inventive Compositions

| Ingredients | Range wt %* |
|---|---|
| hydroxypinacolone retinoate | 0.01%-0.5% |
| Lactic acid or gluconolactone | 1%-5% |
| Eperua falcata bark extract | 0.01%-0.5% |
| Additives/carriers and solvents (e.g., niacinamide, | 0%-20% |
| tocopheryl acetate, tocopherol, hydrolyzed sodium | |
| hyaluronate, linoleic acid (and) linolenic acid, superoxide | |
| dismutase, ubiquinone, pentaerythrityl tetra-di-t-butyl | |
| hydroxyhydrocinnamate, tetrahexyldecyl ascorbate, | |
| palmitoyl tripeptide-1 (and) palmitoyl tetrapeptide-7, | |
| bisabolol, hydrolyzed sodium hyaluronate, camellia | |
| sinensis leaf extract, boron nitride, tetrasodium glutamate | |
| diacetate, sodium hydroxide, sodium PCA, | |
| phenoxyethanol) | |
| Fatty compound (e.g., caprylic/capric triglyceride, | 0.1%-20% |
| butyrospermum parkii butter, glycol palmitate, | |
| cholesterol, squalane, dicaprylyl ether, bis-behenyl/ | |
| isostearyl/phytosteryl dimer dilinoleyl dimer dilinoleate, | |
| C15-19 alkane, dimethicone, dimethicone (and) | |
| polysilicone-11, sodium lauroyl lactylate (and) ceramide | |
| NP (and) ceramide AP (and) phytosphingosine (and) | |
| cholesterol (and) xanthan gum (and) carbomer (and) | |
| ceramide EOP) | |
| Thickener (e.g., xanthan gum, poly C10-30 alkyl | 0.01%-10% |
| acrylate, polyacrylate crosspolymer-6, acrylates/C10-30 | |
| alkyl acrylate crosspolymer) | |
| Surfactant (e.g., glyceryl stearate (and) PEG-100 stearate, | 0.1%-15% |
| steareth-2, steareth-20, methyl gluceth-20, glyceryl | |
| stearate) | |
| Glycerin | 0%-20% |
| Propanediol | 0%-10% |
| Butylene Glycol | 0%-10% |
| Water | 5%--90% |

*each ingredient or combination

Two inventive compositions according to the general formulae shown in Table 2 were prepared according to the following inventive embodiments shown below.

The inventive 1 composition includes hydroxypinacolone retinoate (0.20), lactic acid (2.70), Eperua falcata bark extract (0.10); acrylates/C10-30 alkyl acrylate crosspolymer, xanthan gum, poly C10-30 alkyl acrylate, polyacrylate crosspolymer-6, carbomer (˜1.1%); steareth-2, steareth-20, methyl gluceth-20, glyceryl stearate, PEG-100 stearate (˜5.5%); glycerin, propanediol, butylene glycol, ethylhexylglycerin (˜8%); bis-behenyl/isostearyl/phytosteryl dimer dilinoleyl dimer dilinoleate, Butyrospermum parkii (shea) butter, C15-19 alkane, ceramide ap, ceramide eop, ceramide np, cholesterol, caprylic/capric triglyceride, dicaprylyl ether, dimethicone, dimethicone, Glycine soja (soybean) oil, glycol palmitate, squalane, linoleic acid, linolenic acid, polysilicone-11 (˜17.5%); camellia sinensis leaf extract, citric acid, dextrin, dimethyl isosorbide, bisabolol, boron nitride, hydrolyzed sodium hyaluronate, niacinamide, palmitoyl tetrapeptide-7, palmitoyl tripeptide-1, panthenol, pentaerythrityl tetra-di-t-butyl hydroxyhydrocinnamate, phenoxyethanol, phytosphingosine, polysorbate 20, sodium hydroxide, sodium lactate, sodium lauroyl lactylate, sodium PCA, superoxide dismutase, tetrahexyldecyl ascorbate, tetrasodium glutamate diacetate, tocopherol, tocopherol, tocopherol, tocopherol, tocopheryl acetate, ubiquinone (˜11.5%); and water (˜54%), all amounts as percentage (%) by weight, based on the weight of the composition.

The inventive 2 composition includes hydroxypinacolone retinoate (0.20), lactic acid (2.70), Eperua falcata bark extract (0.10); acrylates/C10-30 alkyl acrylate crosspolymer, xanthan gum, poly C10-30 alkyl acrylate, polyacrylate crosspolymer-6, carbomer (˜1.5%); steareth-2, steareth-20, methyl gluceth-20, glyceryl stearate, PEG-100 stearate (˜8.5%); glycerin, propanediol, butylene glycol, ethylhexylglycerin (˜10%); bis-behenyl/isostearyl/phytosteryl dimer dilinoleyl dimer dilinoleate, Butyrospermum parkii (shea) butter, C15-19 alkane, ceramide ap, ceramide eop, ceramide np, cholesterol, caprylic/capric triglyceride, dicaprylyl ether, dimethicone, dimethicone, Glycine soja (soybean) oil, glycol palmitate, squalane, linoleic acid, linolenic acid, polysilicone-11 (˜8.5%); camellia sinensis leaf extract, citric acid, dextrin, dimethyl isosorbide, bisabolol, boron nitride, hydrolyzed sodium hyaluronate, niacinamide, palmitoyl tetrapeptide-7, palmitoyl tripeptide-1, panthenol, pentaerythrityl tetra-di-t-butyl hydroxyhydrocinnamate, phenoxyethanol, phytosphingosine, polysorbate 20, sodium hydroxide, sodium lactate, sodium lauroyl lactylate, sodium PCA, superoxide dismutase, tetrahexyldecyl ascorbate, tetrasodium glutamate diacetate, tocopherol, tocopherol, tocopherol, tocopherol, tocopheryl acetate, ubiquinone (˜9.5%); and water (˜60%), all amounts as percentage (%) by weight, based on the weight of the composition.

Embodiments of the skin care composition according to the disclosure are considered stable when evaluated for aesthetic texture that is pleasing on application, and as applied to the skin is free of a rough, clingy or sticky feel. Formulation stability is evident, when provided in an emulsion form, wherein the composition does not break or release oily droplets, and when provided in any form does not demonstrate precipitation or breaking into discernible layers.

Embodiments of the skin care composition according to the disclosure are considered a stable composition despite the high amounts of actives when they are shown to have maintained an aesthetically homogeneous phase, wherein there is no visually perceptible signs of phase separation, do not show a grainy texture and/or become inhomogeneous over a period of 8 weeks in a temperature storage range of 4° C. to 45° C., with no significant changes in appearance, pH, viscosity, and color.

### EXAMPLE 3: Cell Studies

### Experiment 1: 2D In Vitro Human Keratinocyte Cell Proliferation and Genetic Analysis

### Rationale & Results

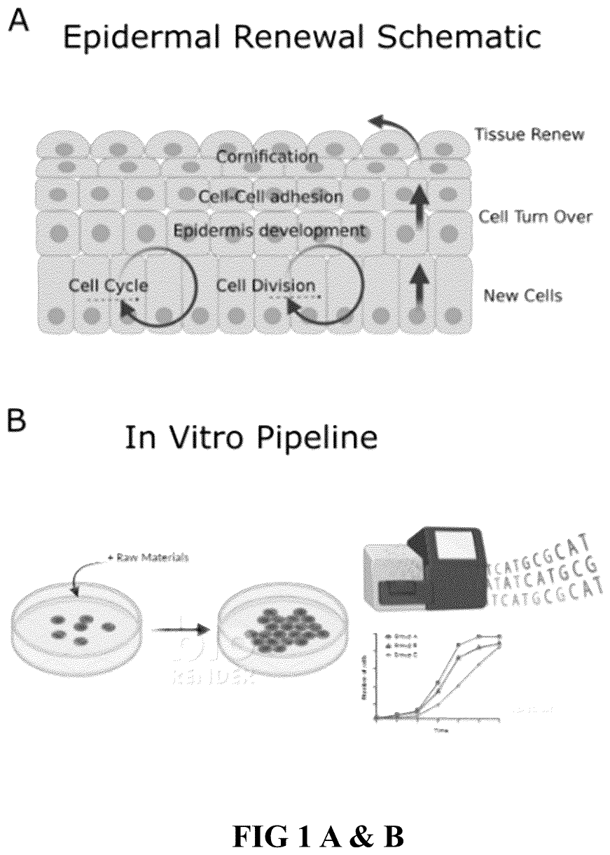

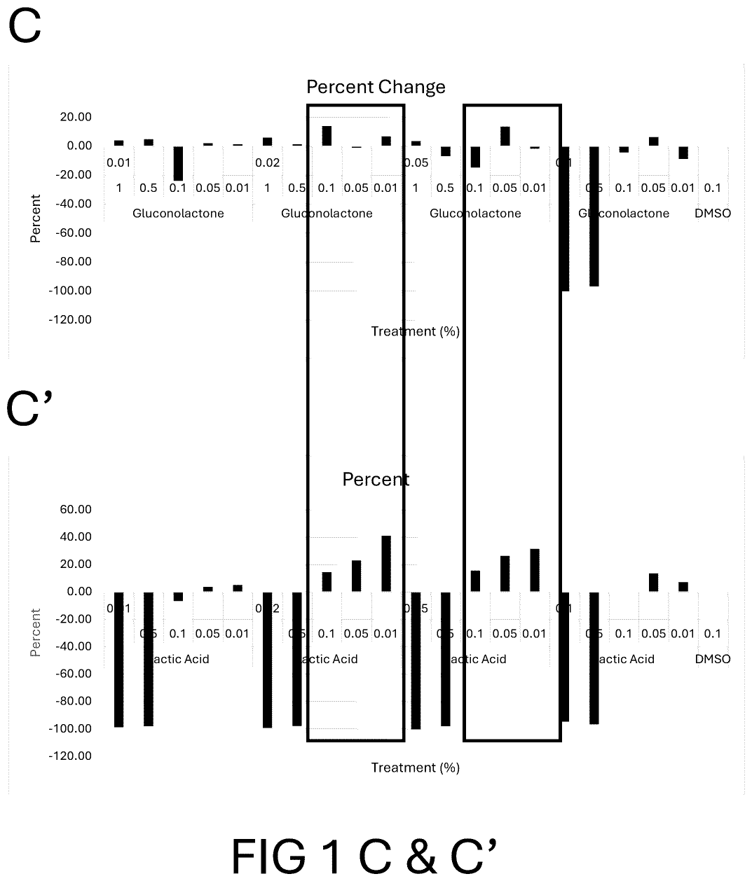

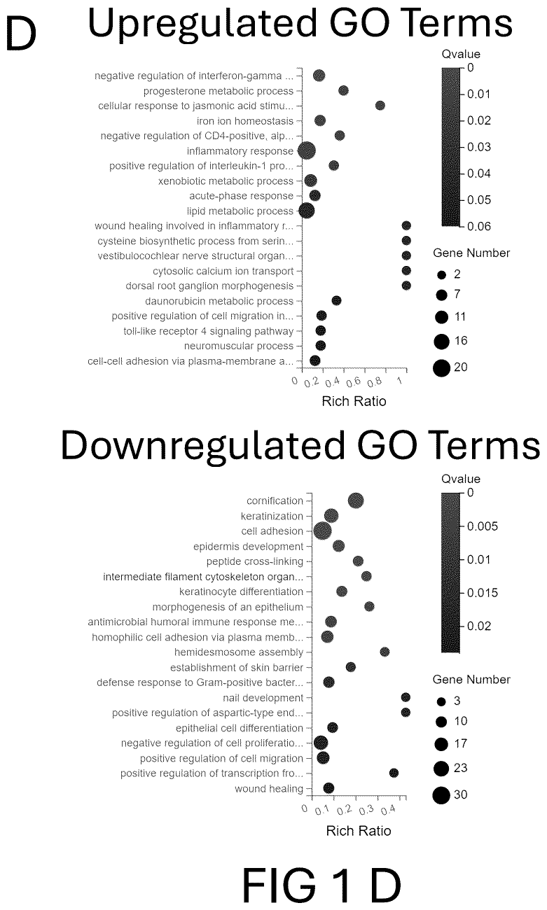

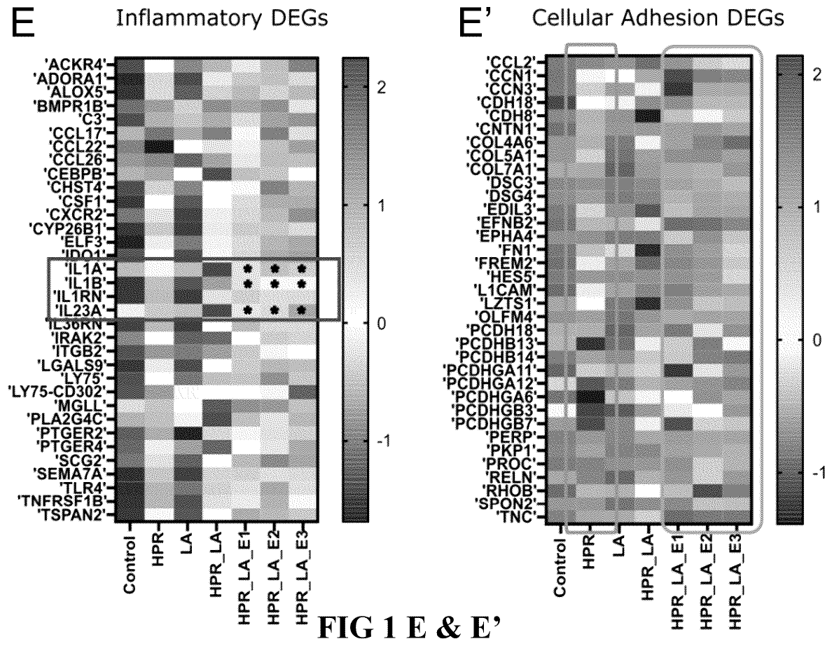

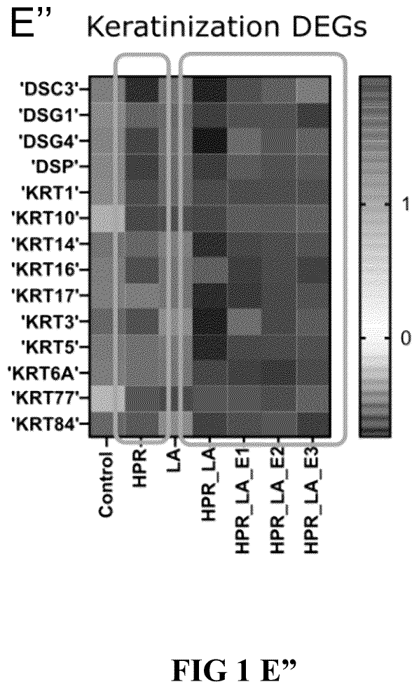

Retinols regenerate the epidermis by stimulating proliferation of keratinocytes which leads to the replacement or turnover of existing cells. Previous genetic analysis indicates this is primarily accomplished via the upregulation of cellular proliferation and migration genes, concomitant with the downregulation of cell-cell adhesion, epidermal barrier and differentiation genes (See FIG. 1A). Given the rapid turnover and shedding of the epithelium, a common side-effect of retinols is inflammation. The inventors characterized the genetic signature of HPR and LA paired with the anti-inflammatory molecule Eperuline, to determine whether these novel combinations exhibited a retinol-based signature with reduced inflammatory markers.

Referring to the drawings, FIG. 1 demonstrates that HPR-LA-Eperuline Combinations stimulate cell proliferation via a retinol genetic signature with anti-inflammatory properties in 2D in vitro Human Keratinocytes. (hydroxypinacolone retinoate is referred to herein as “HPR,” lactic acid is referred to as “LA” and Eperua falcata bark extract is referred to herein as “Eperuline”)

(A) Schematic depicting epidermal turnover, which is characterized by cell division, turnover and renewal. Cell turnover is associated with increased cell proliferation and migration and decreased cell-cell adhesion and mature cell differentiation markers. Inflammation from rapid renewal of the skin occurs.

(B) Schematic of In Vitro Pipeline.

(C,C′) MTT Proliferation Assay; red boxes denote concentrations of HPR and LA which stimulate cell division.

(D). Unbiased Gene Ontology Pathway Analysis of DEGs identified via RNA sequencing between HPR (0.05%)-LA (0.01%) Eperuline (0.01%; 0.05% and 0.1% not shown) treated keratinocytes compared to DMSO controls (0.1%).

(E-E″) Shared DEGS z-scored gene expression between each treatment group. Red boxes and * indicated reduced inflammatory markers with increasing concentrations of Eperuline. Green boxes indicate HPR with Eperuline contains signatures similar to HPR alone.

The inventors evaluated whether HPR paired with alpha-hydroxy (AH) or polyhydroxy (PH) acids stimulated cell proliferation with minimal cellular toxicity.

Human keratinocytes were seeded and cultured overnight prior to a 48-hour treatment (See FIG. 1A′ and Table 3) of increasing concentrations of HPR (0.01%, 0.02%, 0.05% and 0.1%) and either lactic acid or gluconolactone (0.01%, 0.05%, 0.1%, 0.5%, 1%).

TABLE 3
Concentrations of Raw Materials using in 2D intro Keratinocytes

| | 1 | 2 | 3 | 4 |
|---|---|---|---|---|
| Plate 1 | | | | |
| A | DMSO 0.1% | HPR 0.02% | HPR 0.05% | |
| B | DMSO 0.1% | HPR 0.02% | HPR 0.05% | |
| C | DMSO 0.1% | HPR 0.02% | HPR 0.05% | |
| Plate 2 | | | | |
| A | LA 0.01% | HPR 0.02% + 0.01% LA | HPR 0.05% + 0.01% LA | HPR 0.02% + LA 0.01% + |
| | | | | 0.01% E |
| B | LA 0.01% | HPR 0.02% + 0.01% LA | HPR 0.05% + 0.01% LA | HPR 0.02% + LA 0.01% + |
| | | | | 0.01% E |
| C | LA 0.01% | HPR 0.02% + 0.01% LA | HPR 0.05% + 0.01% LA | HPR 0.02% + LA 0.01% + |
| | | | | 0.01% E |
| Plate 3 | | | | |
| A | GL 0.01% | HPR 0.02% + 0.01% GL | HPR 0.05% + 0.01% GL | HPR 0.02% + LA 0.01% + |
| | | | | 0.05% E |
| B | GL 0.01% | HPR 0.02% + 0.01% GL | HPR 0.05% + 0.01% GL | HPR 0.02% + LA 0.01% + |
| | | | | 0.05% E |
| C | GL 0.01% | HPR 0.02% + 0.01% GL | HPR 0.05% + 0.01% GL | HPR 0.02% + LA 0.01% + |
| | | | | 0.05% E |
| Plate 4 | | | | |
| A | HPR 0.02% + LA 0.01% + | HPR 0.02% + GL 0.01% + | HPR 0.02% + GL 0.01% + | HPR 0.02% + GL 0.01% + |
| | 0.1% E | 0.01% E | 0.05% E | 0.1% E |
| B | HPR 0.02% + LA 0.01% + | HPR 0.02% + GL 0.01% + | HPR 0.02% + GL 0.01% + | HPR 0.02% + GL 0.01% + |
| | 0.1% E | 0.01% E | 0.05% E | 0.1% E |
| C | HPR 0.02% + LA 0.01% + | HPR 0.02% + GL 0.01% + | HPR 0.02% + GL 0.01% + | HPR 0.02% + GL 0.01% + |
| | 0.1% E | 0.01% E | 0.05% E | 0.1% E |
| Plate 5 | | | | |
| A | HPR 0.05% + LA 0.01% + | HPR 0.05% + LA 0.01% + | HPR 0.05% + GL 0.01% + | HPR 0.05% + GL 0.01% + |
| | 0.05% E | 0.1% E | 0.05% E | 0.1% E |
| B | HPR 0.05% + LA 0.01% + | HPR 0.05% + LA 0.01% + | HPR 0.05% + GL 0.01% + | HPR 0.05% + GL 0.01% + |
| | 0.05% E | 0.1% E | 0.05% E | 0.1% E |
| C | HPR 0.05% + LA 0.01% + | HPR 0.05% + LA 0.01% + | HPR 0.05% + GL 0.01% + | HPR 0.05% + GL 0.01% + |
| | 0.05% E | 0.1% E | 0.05% E | 0.1% E |
| Plate 6 | | | | |
| A | HPR 0.05% + LA 0.01% + | HPR 0.05% + GL 0.01% + | | |
| | 0.01% E | 0.01% E | | |
| B | HPR 0.05% + LA 0.01% + | HPR 0.05% + GL 0.01% + | | |
| | 0.01% E | 0.01% E | | |
| C | HPR 0.05% + LA 0.01% + | HPR 0.05% + GL 0.01% + | | |
| | 0.01% E | 0.01% E | | |

MTT cell proliferation assays showed that compared to DMSO controls (0.1% DMSO), human keratinocytes exhibited increased cell number following treatment at certain concentration ranges with HPR (0.02% and 0.05%) paired with LA (0.01%, 0.05% & 0.1%) while HPR paired with Gluconolactone did not (See FIG. 1C; red boxes). These data suggested that HPR combined with LA stimulates cell proliferation while HPR paired with gluconolactone diminishes this effect in human keratinocytes.

The inventors treated human keratinocytes with HPR paired with LA at the previously identified concentrations that stimulated cell proliferation (HPR 0.05% and LA 0.01%), with increasing concentrations of Eperuline (0.01%, 0.05%, 0.1%). Following 48-hours of treatment, the inventors performed RNA-sequencing and identified differentially expressed genes (DEGs) between each treatment and control cells. Gene Ontology (GO) pathway analysis of DEGs showed that HPR and LA paired with Eperuline contained a retinol-based genetic signature, as genes with terms associated with inflammation and cell migration were upregulated (See FIG. 1D) while genes associated with epidermal differentiation, adhesion and barrier (See FIG. 1D [Keratinization, Cornification, Epidermis Development, Adhesion] were downregulated. Strikingly, the addition of Eperuline in combination with the HPR-Lactic pairing led to the downregulation of key inflammatory genes in human keratinocytes including IL-1A, IL-1B and IL-23A (FIG. 1E; *) in a dose-dependent manner while retaining the HPR genetic signature (See FIG. 1E′,E″; green boxes). Thus, the unbiased analysis of RNA-sequencing data supports HPR-LA-Eperuline combination as an effective epidermal renewal formula with anti-inflammatory properties.

### Experiment 1 Methods

Human Keratinocytes (HaCat: (AddexBio, cat #T0020001, lot #0003798)) were plated at 160K cells/well in a 12-well plate and allowed to attach overnight in 1 ml of Dulbecco's Modified Eagle Medium (DMEM High Glucose, Gibco) containing Fetal Bovine Serum (10%) and Penicillin Streptomycin (1%, Gibco, 1570063) in a 37° C. incubator (ThermoFisher Scientific, FormaSteri Cycle i160). The following day, cells were treated with 1 ml of tested compounds (Table 3) dissolved in DMEM for 48 hours. MTT Assay Kit (Abcam ab 211091) was then used to assess cellular number and toxicity. Each treatment was replicated 3 times.

For RNA-sequencing experiments, cells were treated as described above. At 48-hours post treatment culture media was removed, followed by a 1×5-min wash with 1×PBS at 37° C. Cells were then trypsinized (Gibco, #15050057) for 5 minutes at 37° C. and transferred to a 1.5 mL Eppendorf tube, lysed in 350 uL RLT Buffer (Qiagen Cat #79216) and stored in 1% BME at −80° C. (2-Mercaptothenol, ThermoFisher Scientific #21985023). Cell lysates were then processed by paired-end bulk-RNA sequencing.

For bulk-RNA sequencing, cell lysates from three biological replicates corresponding to each control or treatment group were combined, which was then followed by RNA extraction with TRIzol reagent (Invitrogen, Thermo Fisher Scientific, Inc) according to standard manufacturer protocols. Each sample underwent library construction and assembly by first treating the samples with DNAse to remove genomic DNA fragments, followed by the isolation of poly-A mRNA transcripts which were then reverse transcribed cDNA libraries. Libraries underwent PCR amplification, followed by 150-paired end sequencing on a HISEQ4000 platform. Raw sequencing reads were filtered and aligned to the Human Reference Genome (GCF_000001405.40_GRCh38.p14) using HISAT and Bowtie software packages. Differentially expressed genes were identified using a Poisson distribution between each treatment group and control DMSO. Gene Ontology analysis was performed using ShinyGOv0.77 software, with z-score gene expression calculated using custom Matlab scripts. Heat Maps were generated using Prism software.

### Experiment 2: Human Ex-Vivo Skin Anti-Inflammatory Analysis

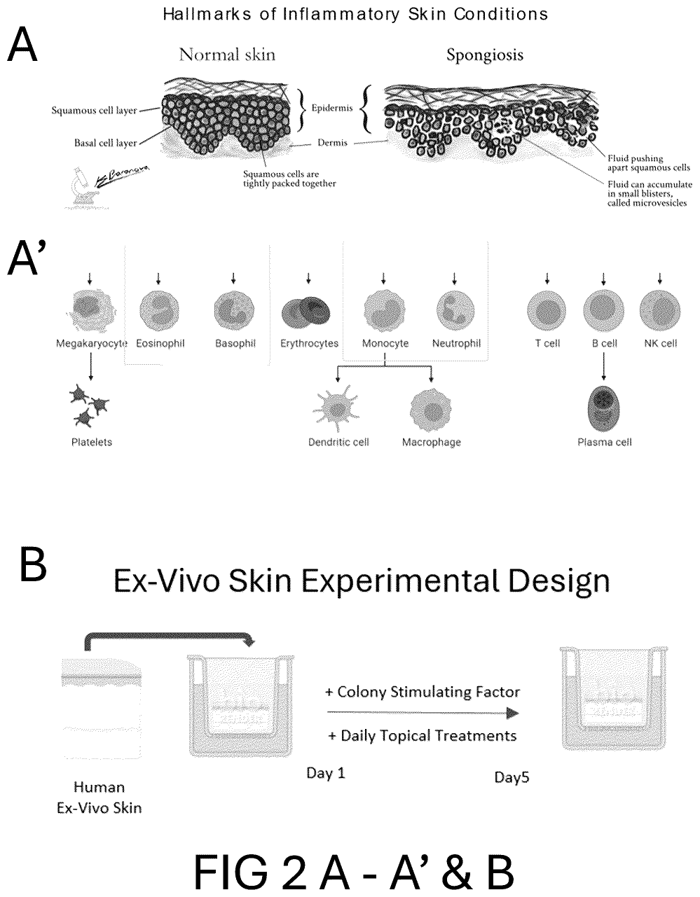

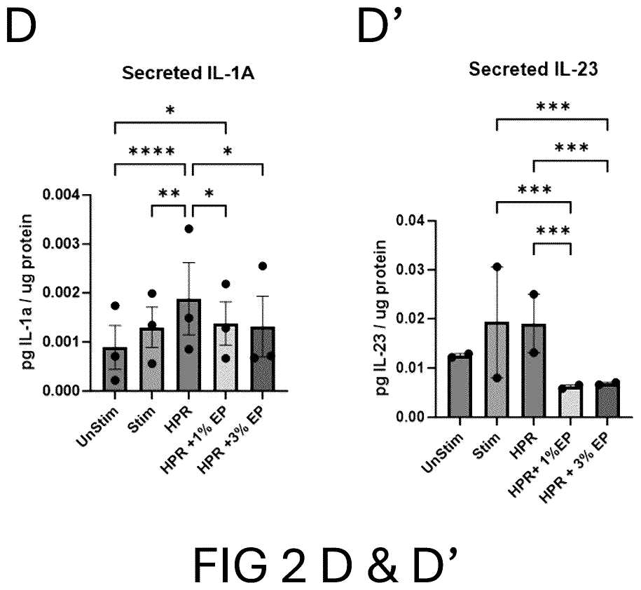

Human inflammatory skin conditions can be modeled within a laboratory setting through the systemic culturing of human ex-vivo skin with colony-stimulating cocktail (CSC). This has been reported to lead to epidermal disruption and infiltration of immune cells akin to inflammatory skin pathologies such as chronic inflammation, atopic dermatitis and psoriasis (See FIG. 2A, A′). Thus, the inventors sought to determine if Eperuline reduced inflammation in models of sensitive skin conditions. Moreover, characterizing the effects of HPR, LA and Eperuline within ex-vivo human skin contains greater physiological relevance compared to in vitro models alone.

Referring again to the drawings, FIG. 2 provides data demonstrating HPR-Eperuline within CSC-stimulated human ex-vivo skin reduces histological signs of inflammation and secreted inflammatory cytokines.

(A-A′) Schematic and diagrams of the hallmarks of skin inflammation and nuclear morphology of infiltrating immune cells (yellow boxes).

(B) Schematic of experimental design.

(C) H&E histological sections of human ex-vivo skin (5 um thickness). Black boxes denote epidermal spongiosis; yellow arrows denote atypical nuclei corresponding to neutrophils/infiltrating immune cells as shown in Figure A′; green arrow indicate parakeratosis.

(D-D″) Quantification of secreted IL-1A and IL-23. Each data point indicates n=3 biological replicates per trial/lot. * indicates significant of p value<0.05, ** indicates significant of p value<0.01, *** indicates significant of p value<0.001, **** indicates significant of p value<0.01.

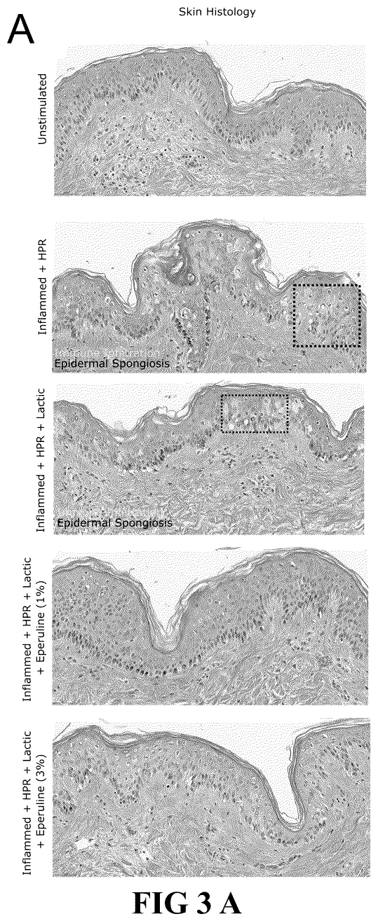

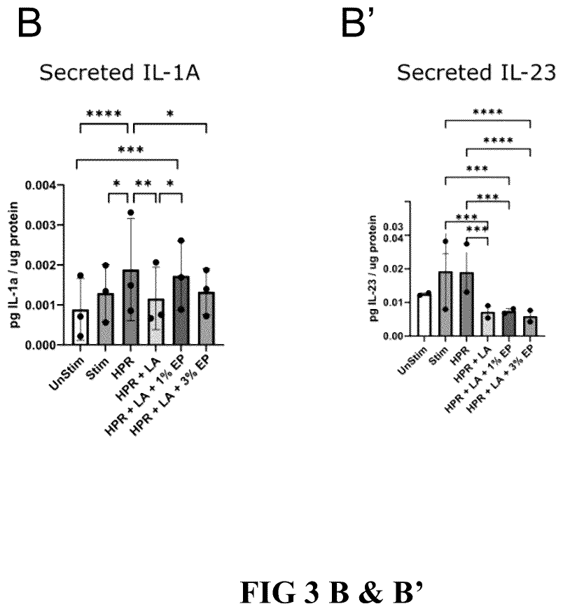

Referring again to the drawings, FIG. 3 provides data demonstrating HPR-LA-Eperuline within CSC-stimulated human ex-vivo skin reduces histological signs of inflammation and secreted inflammatory cytokines.

(A) H&E histological sections of human ex-vivo skin (5 um thickness). Black boxes denote epidermal spongiosis; yellow arrows denote atypical nuclei corresponding to neutrophils/infiltrating immune cells as shown in Figure A′; green arrow indicate parakeratosis.

(B,B′) Quantification of secreted IL-1A and IL-23. Each data point indicates n=3 biological replicates per trial/lot. * indicates significant of p value<0.05, ** indicates significant of p value<0.01, *** indicates significant of p value<0.001, * indicates significant of p value<0.01.

Human ex-vivo skin was cultured with or without CSC and topically treated with either HPR, HPR-LA, HPR-Eperuline and HPR-LA-Eperuline for 5 days consecutive days (See FIG. 2B; See Table 4 for concentrations).

TABLE 4
Concentrations of Raw Materials using in 3D ex-vivo human skin

| Control | HPR | LA | Eperuline |
|---|---|---|---|
| 0.01% EtOH | 0.01% | 3% | 1% |
| | | | 3% |

Histological analysis showed that compared to controls, CSC-stimulated human skin exhibited hallmarks of inflammation including epidermal spongiosis (FIG. 2A, C; boxes), which was exacerbated with topical treatments of HPR (See FIG. 2C; boxes). Consistent with increased inflammation following HPR treatments, the inventors identified infiltrating immune cells via nuclear morphology (See FIG. 1A; yellow boxes, See FIG. 2C; yellow arrow), parakeratosis (See FIG. 2C; green arrow) and a significant increase in secreted inflammatory cytokines, such as IL-1A and IL-23 (See FIG. 2D-D″).

Strikingly, the addition of Eperuline with HPR or HPR-lactic acid led to reduced histological signs of inflammation such as epidermal spongiosis and immune infiltration (See FIG. 2C, 3A), accompanied by a significant reduction in secreted IL-1A and IL-23 (See FIG. 2D,D′, FIG. 3B,B′). Notably, both IL-1A and IL-23 were transcriptionally downregulated in the previous RNA-sequencing experiments following Eperuline treatment, and represent inflammatory cytokines associated with skin sensitization and chronic inflammation. Thus, these collective results are suggestive that combinations of HPR with either AH acids and/or Eperuline maintain the benefits of retinol-based epidermal renewal while reducing inflammation and could be used to treat age-related skin decline in sensitized skin populations.

### Experiment 2 Methods

Fresh human ex-vivo skin was obtained through donor informed consent following abdominoplasty procedures. The tissues were obtained through BioIVT (Westbury, NY, USA) following an approved IRB protocol and shipped to a BSC level 2 research laboratory within 24 h after the procedure where the experiments took place. All experiments were performed in accordance with relevant guidelines and regulations. Donors included a 38 year old African American Female, 54 year old Hispanic Female and 63 year old Caucasian Female. Ex vivo tissues were prepared by excising the subcutaneous fat followed by punch biopsies with disposable 12 mm Skin Biopsy Punch (Fisher Scientific, NC9253254) and placed in 24-well transwell plates with permeable polyester membrane inserts (Fisher Scientific, 07-200-161). At least three biopsies were used for each condition.

Skin tissues were divided into groups of three and placed in the 24-well transwell plates, colony stimulant factor (Cell stimulation cocktail 500×(50-930-5)) was diluted to a 2× concentration in culture media and 650 uL was added to the well on the first day. Topical treatments were undertaken daily by pipetting 5 uL of treatments onto the tissue and using a Q-tip, spreading treatments across the tissue and left to absorb at room temperature for 30 minutes. Base media without stimulants served as the control group. Tissues were replenished with fresh media with the stimulant on day 1 and 3. On days 1, 3 and 5, supernatant from the culture was collected and aliquoted, and the tissues were bisected and fixed in 10% neutral buffered formalin (Fisher Scientific 22-126-347) for 3 h and then switched to 70% ethanol for storage until histology.

Skin tissues were processed into formalin-fixed, paraffin-embedded (FFPE) block following a standard protocol (Histowiz, Inc., Long Island City, NY, USA) prior to histological staining. Hematoxylin and eosin (H&E) staining was employed as an analytical tools to elucidate the impact on the epidermal tissue structure with standard protocols.

Tissue culture supernatant was collected at the end of the cultures to evaluate the secretion of several cytokines, chemokines, and growth factors. A 13-plex inflammatory cytokine and a 10-plex angiogenesis ProcartaPlex panel were used in our experiments (ThermoFisher, Waltham, MA, USA). Sample preparation was carried out following the manufacturer's protocol and scanned using a Luminex MAGPIX Instrument System (ThermoFisher). The supernatant from each tissue was run in duplicate wells with at least two tissues from each condition for each donor and with at least three donors.

Data were assessed using a two-way ANOVA with multiple comparison test. The differences between groups were compared using GraphPad Prism 9.0.1 (GraphPad Software Inc., La Jolla, CA, USA)

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1) A skin care composition comprising a plurality of agents that include at least one retinol derivative and at least one bark extract or plant extract with a high concentration of dihydroflavonols and catechuic tannins, the plurality of agents providing reduced retinol associated inflammation as compared with a composition that includes the at least one retinol and lacks the at least one bark extract, wherein the plurality of agents is selected from the group consisting of:
i. at least one retinol derivative comprising hydroxypinacolone retinoate and at least one bark extract or plant extract with a high concentration of dihydroflavonols and catechuic tannins;
ii. at least one retinol derivative comprising hydroxypinacolone retinoate, at least one bark extract or plant extract with a high concentration of dihydroflavonols and catechuic tannins, and at least one hydroxy acid; and
iii. at least one retinol derivative comprising hydroxypinacolone retinoate, at least one bark extract comprising Eperua falcata bark extract, and at least one hydroxy acid comprising lactic acid,
wherein the composition confers one or more of increased skin cell proliferation, improved cellular migration, improved cellular differentiation, or a combination thereof, and wherein application the plurality of agents confers to skin cells and tissues an anti-inflammatory retinol genetic signature as demonstrated by via bulk-RNA sequencing of human keratinocytes and restores epidermal disruption and reduce IL-1A and IL-23 inflammatory cytokines in human ex-vivo skin models of chronic inflammation.

2) A method for minimizing the signs of aging in skin, the method comprising:
(A) providing a plurality of agents that include at least one retinol derivative and at least one bark extract, the plurality of agents providing reduced retinol associated inflammation as compared with a composition that includes the at least one retinol and lacks the at least one bark extract, wherein the plurality of agents is selected from the group consisting of:
i. at least one retinol derivative comprising hydroxypinacolone retinoate and at least one bark extract;
ii. at least one retinol derivative comprising hydroxypinacolone retinoate, at least one bark extract, and at least one hydroxy acid; and
iii. at least one retinol derivative comprising hydroxypinacolone retinoate, at least one bark extract comprising Eperua falcata bark extract, and at least one hydroxy acid comprising lactic acid,
wherein the composition confers one or more of increased skin cell proliferation, improved cellular migration, improved cellular differentiation, or a combination thereof;
(B) applying the composition to skin; and
(C) following a regimen of repeated application of the composition for a period of at least 1 day,
wherein application the plurality of agents confers to skin cells and tissues an anti-inflammatory retinol genetic signature as demonstrated by via bulk-RNA sequencing of human keratinocytes and restores epidermal disruption and reduce IL-1A and IL-23 inflammatory cytokines in human ex-vivo skin models of chronic inflammation.

3) The method according to claim 2, wherein the method includes providing one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

4) A skin care composition, comprising:
an anti-aging system, comprising:
i. at least one retinol derivative;
ii. at least one hydroxy acid; and
iii. at least one bark extract.

5) The composition according to claim 4, wherein in the anti-aging system:
i. the at least one retinol derivative is selected from the group consisting of hydroxypinacolone retinoate, retinol, retinyl acetate, retinyl palmitate, retinyl propionate, retinyl retinoate, bakuchiol, and combinations thereof;
ii. the at least one hydroxy acid is selected from the group consisting of lactic acid, gluconolactone, glycolic acid, mandelic acid, salicylic acid, and combinations thereof; and
iii. the at least one bark extract is selected from the group consisting of Eperua falcata bark extract, pine bark extract (Pinus pinaster bark/bud extract), Ficus regligiosa bark, Witch Hazel Extract, a plant extract with a high concentration of dihydroflavonols and catechuic tannins, and combinations thereof.

6) The composition according to claim 4, wherein in the anti-aging system:
i. the at least one retinol derivative comprises hydroxypinacolone retinoate;
ii. the at least one hydroxy acid comprises lactic acid or gluconolactone; and
iii. the at least one bark extract comprises Eperua falcata bark extract.

7) The composition according to claim 4 wherein, in the anti-aging system;
i. the at least one retinol derivative is present from about 0.001% to about 3.0%, ii. the at least one hydroxy acid is present from about 0.001% to about 5.0%; and
iii. the at least one bark extract is present from about 0.0001% to about 70%,
all amounts by weight, based on the total weight of the composition.

8) The composition according to claim 4 wherein in the anti-aging system;
i. the at least one retinol derivative comprises hydroxypinacolone retinoate present from about 0.05% to about 0.5%;
ii. the at least one hydroxy acid comprises lactic acid present from about 1.0% to about 5.0%; and
iii. the at least one bark extract comprises Eperua falcata bark extract present from about 0.05% to about 0.5%,
all amounts by weight, based on the total weight of the composition.

9) The composition according to claim 8 wherein in the anti-aging system:
i. hydroxypinacolone retinoate is present at about 0.1%;
ii. lactic acid is present at about 3%; and
iii. Eperua falcata bark extract is present at about 0.1%,
all amounts by weight, based on the total weight of the composition.

10) The composition according to claim 4, comprising:
a carrier system comprising:
i. at least one surfactant;
ii. at least one fatty compound; and
iii. at least one polymeric thickener.

11) The composition according to claim 10, wherein the at least one surfactant is selected from the group consisting of glyceryl stearate (and) PEG-100 stearate, steareth-2, steareth-20, methyl gluceth-20, glyceryl stearate, and combinations thereof, the at least one fatty compound is selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof, and the at least one polymeric thickener is selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof.

12) The composition according to claim 10, the carrier system comprising:
i. water, a water-based solvent, or a combination thereof; and
ii. optionally, one or more additives selected from the group consisting of niacinamide, tocopherol, tocopheryl acetate, tocopherol, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, tetrasodium glutamate diacetate, sodium hydroxide, sodium PCA, phenoxyethanol, and combinations thereof.

13) A skin care composition, comprising:
an anti-aging system, comprising:
i. at least one retinol derivative;
ii. at least one hydroxy acid; and
iii. at least one bark extract; and
a carrier system comprising:
i. at least one surfactant;
ii. at least one fatty compound;
iii. at least one polymeric thickener;
iv. water, a water-based solvent, or a combination thereof; and
v. optionally, one or more additives.

14) The composition according to claim 13 wherein:
in the anti-aging system:
i. the at least one retinol derivative is selected from the group consisting of hydroxypinacolone retinoate, retinol, retinyl acetate, retinyl palmitate, retinyl propionate, retinyl retinoate, bakuchiol, and combinations thereof;
i. the at least one hydroxy acid is selected from the group consisting of lactic acid, gluconolactone, glycolic acid, mandelic acid, salicylic acid, and combinations thereof; and
ii. the at least one bark extract is selected from the group consisting of Eperua falcata bark extract, pine bark extract (Pinus pinaster bark/bud extract), Ficus regligiosa bark, Witch Hazel Extract, a plant extract with a high concentration of dihydroflavonols and catechuic tannins, and combinations thereof; and
in the carrier system:
i. the at least one surfactant is selected from the group consisting of glyceryl stearate (and) PEG-100 stearate, steareth-2, steareth-20, methyl gluceth-20, glyceryl stearate, and combinations thereof;
ii. the at least one fatty compound is selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof; and
iii. the at least one polymeric thickener is selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof.

15) The composition according to claim 13, comprising at least one additive selected from the group consisting of niacinamide, tocopherol, tocopheryl acetate, tocopherol, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, tetrasodium glutamate diacetate, sodium hydroxide, sodium PCA, phenoxyethanol, and combinations thereof.

16) The composition according to claim 13, wherein in the carrier system:
i. the composition comprises water present in a range from about from about 25% to about 75%;
ii. the at least one surfactant is present in a range from about 0.5% to about 3%;
iii. the at least one fatty compound is present in a range from about 0.1% to about 20%; and
iv. the at least one polymer thickener is present in a range from about 0.01% to about 10%,
all amounts by weight, based on the total weight of the composition.

17) A skin care composition comprising:
an anti-aging system, comprising:
i. at least one retinol derivative comprising hydroxypinacolone retinoate present from about 0.05% to about 0.5%; and
ii. at least one bark extract comprising Eperua falcata bark extract present from about 0.05% to about 0.5%,
all amounts by weight, based on the total weight of the composition.

18) The composition according to claim 17, comprising:
a carrier system comprising:
i. at least one surfactant;
ii. at least one fatty compound; and
iii. at least one polymeric thickener.

19) The composition according to claim 18, wherein the at least one surfactant is selected from the group consisting of glyceryl stearate (and) PEG-100 stearate, steareth-2, steareth-20, methyl gluceth-20, glyceryl stearate, and combinations thereof, the at least one fatty compound is selected from the group consisting of caprylic/capric triglyceride, Butyrospermum parkii butter, glycol palmitate, cholesterol, squalane, dicaprylyl ether, bis-behenyl/isostearyl/phytosteryl dimer dilinoleyl dimer dilinoleate, C15-19 alkane, dimethicone, dimethicone (and) polysilicone-11, sodium lauroyl lactylate (and) ceramide NP (and) ceramide AP (and) phytosphingosine (and) cholesterol (and) xanthan gum (and) carbomer (and) ceramide EOP, and combinations thereof, and the at least one polymeric thickener is selected from the group consisting of xanthan gum, poly C10-30 alkyl acrylate, polyacrylate crosspolymer-6, acrylates/C10-30 alkyl acrylate crosspolymer, sclerotium gum, acacia gum, carbomer, acrylate crosspolymer, hydroxyethyl cellulose, cellulose, and combinations thereof.

20) The composition according to claim 18, the carrier system comprising:
i. water, a water-based solvent, or a combination thereof; and
ii. optionally, one or more additives selected from the group consisting of niacinamide, tocopherol, tocopheryl acetate, tocopherol, panthenol, hydrolyzed sodium hyaluronate, linoleic acid (and) linolenic acid, superoxide dismutase, ubiquinone, pentaerythrityl tetra-di-t-butyl hydroxyhydrocinnamate, tetrahexyldecyl ascorbate, palmitoyl tripeptide-1 (and) palmitoyl tetrapeptide-7, bisabolol, hydrolyzed sodium hyaluronate, camellia sinensis leaf extract, boron nitride, tetrasodium glutamate diacetate, sodium hydroxide, sodium PCA, phenoxyethanol, and combinations thereof.
